(19) United States
(12) Patent Application Publication
(10) Pub. No.: US 2026/0305914 A1
(43) Pub. Date: Oct. 8, 2026

(54) DISTAL TIP FOR MEDICAL DEVICES AND RELATED METHODS

(71) Applicant: Boston Scientific Scimed, Inc., Maple Grove, MN (US)

(72) Inventors: Abdiqadar Ahmed MOHAMUD, Blaine, MN (US); Newell G. VOSS, St Paul, MN (US); Jack KOLLWITZ, Bloomington, MN (US); Alex MADZIA, Columbus, OH (US); Neal ANDERSON, Northborough, MA (US)

(73) Assignee: Boston Scientific Scimed, Inc., Maple Grove, MN (US)

(21) Appl. No.: 19/462,389

(22) Filed: Jan. 28, 2026

Related U.S. Application Data
(60) Provisional application No. 63/751,031, filed on Jan. 29, 2025.

(51) Int. Cl. A61B 1/00; A61B 1/015; A61B 1/05; A61B 1/06; A61B 1/273

(52) U.S. Cl. CPC A61B 1/0008; A61B 1/0011; A61B 1/015; A61B 1/05; A61B 1/06; A61B 1/2736

(57) ABSTRACT

A distal tip of a medical device includes a housing, a dam, and an electronics assembly. The housing may define a distal opening of a working channel. The dam may have a lateral wall and a distal wall. In an unassembled state of the distal tip, the dam may be separate from the housing. In the assembled state of the distal tip, the dam and the housing may define a chamber. The electronics assembly may be disposed within the chamber in the assembled state of the distal tip. The electronics assembly may include an imager and a lightning element. The distal tip includes an encapsulation material. The encapsulation material may be within the chamber and may at least partially surround the electronics assembly.

### CROSS-REFERENCE TO RELATED APPLICATIONS

This application claims the benefit of priority to U.S. Provisional Application No. 63/751,031, filed on Jan. 29, 2025, which is incorporated by reference herein in its entirety.

### TECHNICAL FIELD

Various aspects of this disclosure relate generally to distal tips of medical devices. In particular, aspects of this disclosure pertain to distal tips and distal tip features of duodenoscopes or other types of medical devices, among other aspects.

### BACKGROUND

A duodenoscope or other type of medical device may include a handle and a sheath, and the sheath may be insertable into a body lumen of a subject. The sheath may terminate in a distal tip portion, which may include features such as optical or other electronic elements (e.g., imager, lighting), air/water outlets, and working channel openings. An elevator may be disposed at the distal tip portion and may be actuatable in order to change an orientation of an accessory device passed through the working channel. For example, the elevator may be pivotable or otherwise movable. Elements in the handle of the duodenoscope may control the elements of the distal tip of the duodenoscope. For example, buttons, knobs, levers, etc. may control elements of the distal tip.

### SUMMARY

Each of the aspects disclosed herein may include one or more of the features described in connection with any of the other disclosed aspects. Aspects of the disclosure may relate to side-facing distal tips of medical devices having housing components to receive elements before an encapsulation or overmolding material is incorporated into the distal tip.

In an example, a distal tip of a medical device comprises a housing and a dam. The housing may define a distal opening of a working channel. The dam may have a lateral wall and a distal wall. In an unassembled state of the distal tip, the dam may be separate from the housing. In an assembled state of the distal tip, the dam and the housing may define a chamber. The distal tip may further comprise an electronics assembly. The electronics assembly may be disposed within the chamber in the assembled state of the distal tip. The electronics assembly may include an imager and a lighting element. The distal tip may comprise an encapsulate or encapsulation material within the chamber and at least partially surrounding the electronics assembly.

Any of the aspects disclosed herein may have any of the following features, alone or in any combination. The dam may include a protrusion that is received by a recess of the housing in the assembled state. In the assembled state, a proximal portion of the electronics assembly may extend into a channel of the housing. The channel may have a smaller cross-section of the imager. The dam may have a substantially “L”-shape. The chamber may have a top opening. The imager and the lighting element may face outward, through the top opening. The housing may include a ramp positioned proximal of the chamber. The housing may define a fluid channel opening positioned above the chamber. The ramp may be configured to guide a fluid of fluid from the fluid channel opening across a lens of the imager or across the encapsulation material. The ramp may include an angled surface that is angled upward in a distal direction. The dam may include a recess at least partially aligned with the imager.

The housing may include a protrusion. The protrusion may at least partially define the chamber. The protrusion may include a base and one or more side walls. A side wall of the one or more side walls may include a first recess at least partially aligned with the imager. A side wall of the one or more side walls includes a second recess at least partially aligned with the lighting element. A proximal end of the base may include a recess. The protrusion may be a first protrusion. The housing may include a second protrusion. The first protrusion and the second protrusion may define a channel therebetween for an elevator.

In another aspect of this disclosure, a distal tip for a medical device comprises a housing and a dam. The housing may include a distal face. The distal tip may further comprise a first protrusion and a second protrusion. The first protrusion and the second protrusion may extend distally from the distal face. The dam may be coupled to the second protrusion. The distal tip may comprise an electronics assembly positioned within an electronics chamber. The dam and the second protrusion may together define boundaries of the electronics chamber.

Any of the aspects disclosed herein may have any of the following features, alone or in any combination. For example, the second protrusion may include a base surface and one or more side walls extending from the base surface. The dam may include one or more walls. At least one of the one or more walls of the dam may include a first feature configured to engage with a second feature of the one or more side walls of the second protrusion to form the boundaries of the electronics chamber.

In an aspect of this disclosure, a method for manufacturing a distal tip of a medical device is disclosed. The method may include inserting an electronics assembly into a housing of the distal tip of the medical device. The method may further include forming an electronics chamber around a distal portion of the electronics assembly by coupling a dam to the housing. The method may include filling the electronics chamber with an encapsulation material. The method may further include curing the encapsulation material.

Any of the aspects disclosed herein may have any of the following features, alone or in any combination. Forming the electronics chamber may comprise coupling the dam to a protrusion of the distal tip. The encapsulation material may form a fluid-tight seal between at an opening of the electronics chamber and an opening of an electronics channel of the housing.

### BRIEF DESCRIPTION OF THE FIGURES

The accompanying drawings are incorporated in and constitute a part of this specification. These drawings illustrate aspects of the disclosure that, together with the written descriptions herein, serve to explain this disclosure. Each drawing depicts one or more exemplary aspects according to this disclosure, as follows:

FIG. 1 depicts an exemplary medical device.

FIG. 2 depicts a perspective view of a portion of an exemplary distal tip for use with the medical device of FIG. 1.

FIG. 3 depicts a partially-exploded perspective view of the distal tip of FIG. 2.

FIG. 4 depicts a perspective view of an electronics assembly of the distal tip of FIG. 2.

FIG. 5 depicts a perspective view of a housing of the distal tip of FIG. 2.

FIGS. 6A-6C depict a portion of the housing of the distal tip of 5 in a perspective view (FIG. 6A), a side view (FIG. 6B), and a top view (FIG. 6C).

FIGS. 7A-7B depict another portion of the housing of the distal tip of FIG. 5 in a first perspective view showing an interior surface of the portion (FIG. 7A) and in a second perspective view showing an exterior surface of the portion (FIG. 7B).

FIG. 8 depicts a flowchart of a method of manufacturing the distal tip of FIG. 2.

### DETAILED DESCRIPTION OF THE FIGURES

Reference will now be made in detail to examples of the present disclosure described above and illustrated in the accompanying drawings. Wherever possible, the same reference numbers will be used throughout the drawings to refer to the same or like parts.

The terms “proximal” and “distal” are used herein to refer to the relative positions of the components of an exemplary medical device. When used herein, “proximal” refers to a position relatively closer to the exterior of the body or closer to a medical professional using the medical device. In contrast, “distal” refers to a position relatively further away from the medical professional using the medical device, or closer to the interior of the body. As used herein, the terms “comprises,” “comprising,” “has,” “having,” “includes,” “including,” or other variations thereof, are intended to cover a non-exclusive inclusion, such that a device or method that comprises a list of elements does not include only those elements, but may include other elements not expressly listed or inherent thereto. Further, as used herein, the term “or” indicates non-exclusive inclusion, such that a list of elements includes one or more, or all, of the listed elements unless otherwise indicated. Unless stated otherwise, the term “exemplary” is used in the sense of “example” rather than “ideal.” As used herein, the terms “about,” “substantially,” and “approximately,” indicate a range of values within +/- 10% of a stated value.

A duodenoscope or other medical device may be utilized to perform a medical procedure at a treatment site within a body lumen of a subject. In procedures such as endoscopic retrograde cholangiopancreatography (ERCP), an operator may navigate the medical device to a duodenum of a subject, and utilize an elevator of a distal tip of the medical device to change an orientation of one or more accessory devices (e.g., tomes, guidewires, or the like) in order to, e.g., cannulate a papilla of the subject.

The distal tip of the medical device may house electronics, outlets for fluidics (e.g., air and water) lumens, or an elevator. In at least some disclosed aspects, the distal tip may include a housing (e.g., a frame or a core) that includes a primary portion and a secondary portion. Electronic components of the distal tip may be installed into the primary portion, and the secondary portion may be affixed to the primary portion to enclose an outer side of the housing. An encapsulation material or overmold (e.g., epoxy) may be inserted into a gap created by a space between the primary and secondary portions of the housing. The secondary portion may serve as a dam to enclose the encapsulation/overmolded material.

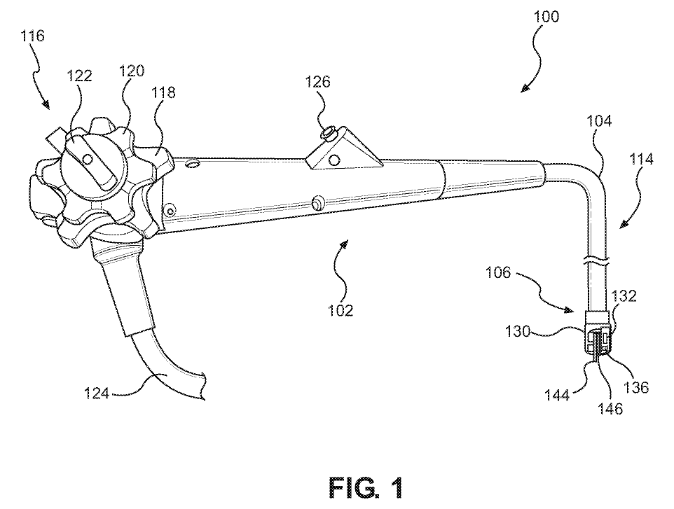

FIG. 1 depicts an exemplary medical device 100 having a handle 102 and an insertion portion 114. Medical device 100 may also include an umbilicus 124 for purposes of connecting medical device 100 to sources of, for example, air, water, suction, power, etc., as well as to image processing and/or viewing equipment. Although duodenoscopes are particularly referenced herein, the disclosure also encompasses other types of devices, such as endoscopes, bronchoscopes, gastroscopes, EUS scopes, colonoscopes, ureteroscopes, bronchoscopes, laparoscopes, cystoscopes, aspiration scopes, sheaths, catheters, or similar devices.

Insertion portion 114 may include a sheath or shaft 104 and a distal tip 130. Shaft 104 may include a plurality of lumens (not shown) extending from a proximal end of shaft 104 to a distal end 106 of shaft 104. For example, the plurality of lumens may include a working channel, fluid delivery channels (e.g., air and water), and a sheath enclosing wiring/cabling for electronics. Distal tip 130 may include an imager 132 (e.g., a camera) and a lighting element 136 (e.g., a light emitting diode (LED) or an optical fiber). Although the term "lighting element" is used herein, it will be appreciated that the term "lighting element" may include a plurality of lighting elements (e.g., a plurality of LEDs or optical fibers). Distal tip 130 may be side-facing. That is, imager 132 and lighting element 136 may face radially outward, perpendicularly, approximately perpendicularly, or otherwise transverse to a longitudinal axis of shaft 104 and distal tip 130 (e.g., at an angle of approximately 80 degrees to an angle of approximately 110 degrees). However, the disclosure is not limited to such an arrangement.

Distal tip 130 may further include an elevator 146 at a distal end of the working channel of the medical device 100, for changing an orientation of an accessory device or tool 144 inserted in the working channel of medical device 100. Elevator 146 may alternatively be referred to as a swing stand, pivot stand, raising base, or any suitable other term.

A distal portion of shaft 104 that is connected to distal tip 130 may have a steerable section. Steerable section may include, for example, an articulation joint. Shaft 104 and the steerable section may include a variety of structures which are known or may become known in the art. Handle 102 may have one or more actuators/control mechanisms 116. One or more of the control mechanisms 116 may provide control over the steerable section or may allow for provision of air, water, suction, etc. For example, handle 102 may include control knobs 118, 120 for left, right, up, and/or down control of the steerable section. For example, one of knobs 118, 120 may provide left/right control of the steerable section and the other of knobs 118, 120 may provide up/down control of the steerable section. Handle 102 may further include one or more locking mechanisms 122 (e.g. knobs or levers) for preventing steering and/or braking of the steerable section in at least one of an up, down, left, or right direction.

One or more of control mechanisms 116 may include one or more elevator control levers (not shown). The lever may control (e.g., be coupled to) a cable or wire connected to the lever. The cable or wire connected to the lever may be directly or indirectly connected to a portion of elevator 146 or another suitable structure. Actuating levers may move one or more cables proximally or distally and thereby actuate elevator 146, as described in further detail below.

In use, an operator may insert at least a portion of shaft 104 into a body lumen of a subject. Distal tip 130 may be navigated to a procedure site in the body lumen. For example, the operator may push or urge distal tip 130 distally in order to advance distal tip 130 through the body lumen. The operator may insert accessory device 144 () into port 126, and pass the accessory device through shaft 104 via a working channel to distal tip 130. Although a distal end of accessory device 144 is labeled, a proximal portion of accessory device 144 extending proximally from port 126, is not show for ease of illustration. The accessory device may exit the working channel at distal tip 130. The user may use one or more of the elevator control levers (not shown) to raise elevator 146 and angle the accessory device toward a desired location (e.g., a papilla of the pancreatico-biliary tract). The user may use the accessory device to perform a medical procedure.

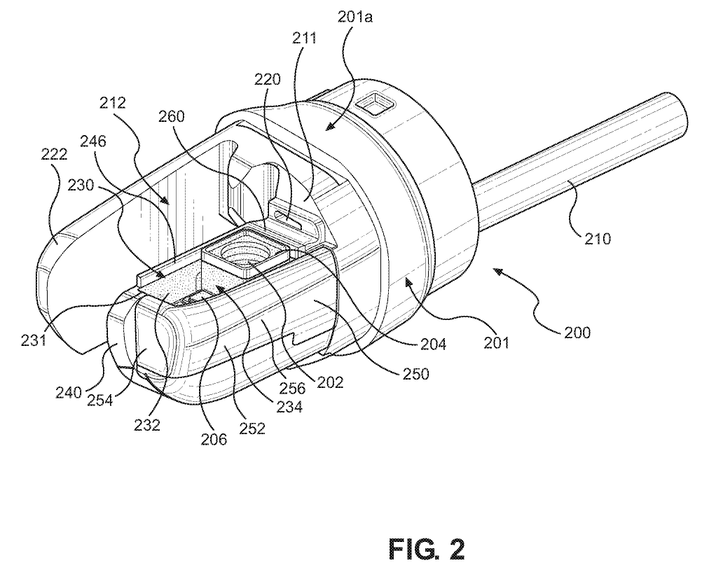

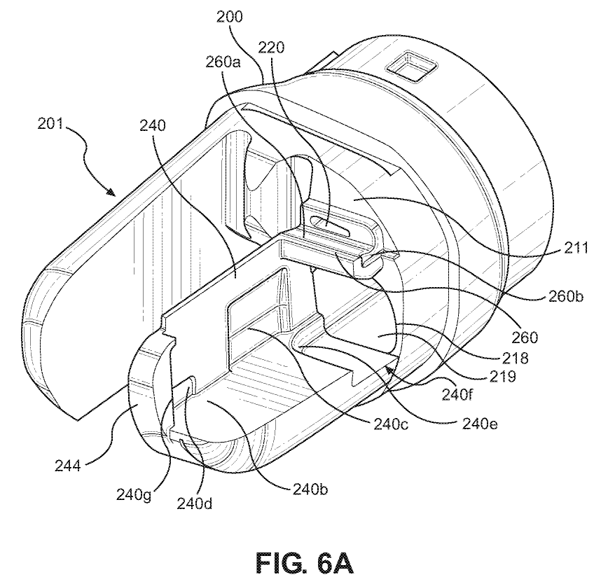

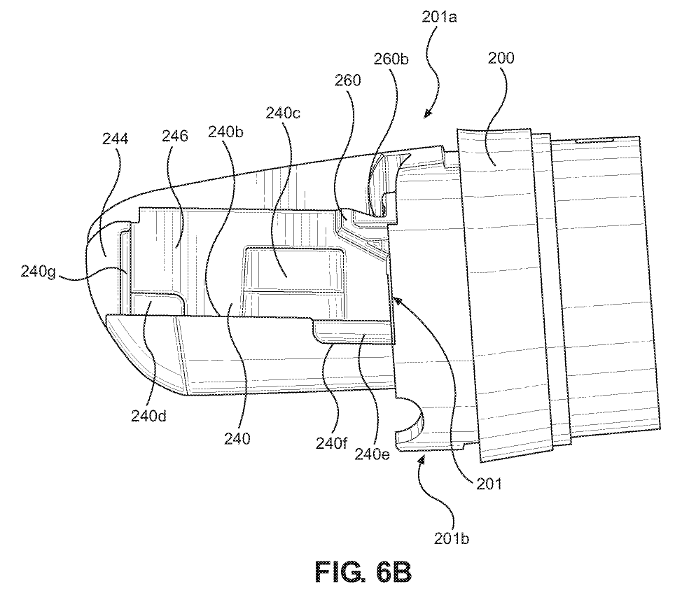

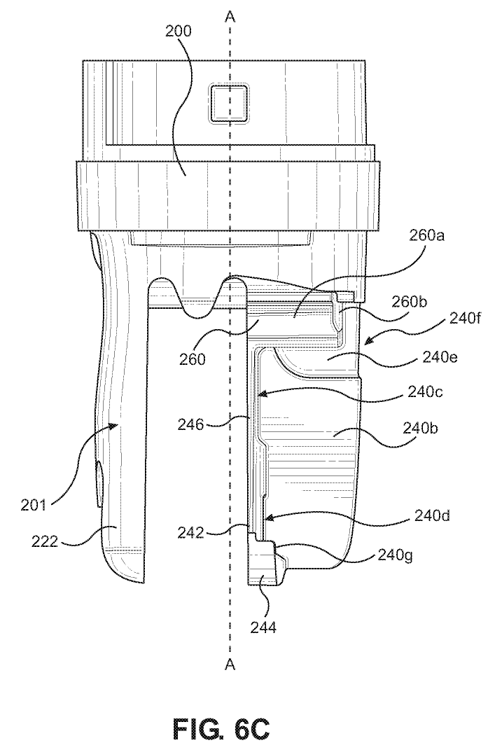

FIGS. 2-7B depict aspects of a distal tip 200, and components thereof, in various configurations. Distal tip 200 may be used instead of distal tip 130 with medical device 100 and may have any of the features of distal tip 130, and vice versa. Certain elements of distal tip 200 (e.g. caps, covers, sheaths, etc.) may be omitted from FIGS. 2-7B to more clearly illustrate aspects of the disclosure. For example, a cap having any property of caps known in the art may be fitted over the elements of distal tip that are shown in FIG. 2. Distal tip 200 may include a housing 201. Housing 201 may be a core, scaffold, or frame for receiving elements of distal tip 200. Housing 201 may include a top side 201a (or top portion) and a bottom side 201b (or bottom portion) (FIG. 6B). Accordingly, relational terms such as 'above' or 'below' may be used in reference to top side 201a and bottom side 201b. For example, the term 'above' may refer to a position relative to a component that is facing, adjacent to, or more proximate to top side 201a and the term 'below' may refer to a position relative to a component that is facing, adjacent to, or more proximate to bottom side 201b.

Housing 201 may include a first protrusion 222 and a second protrusion 240. The first protrusion 222 and the second protrusion 240 may extend distally from a distal face 211 of a proximal portion of housing 201. Each of first protrusion 222 and second protrusion 240 may be integrally formed with a remainder of housing 201, and each may be formed from a plastic, polymer, or similar material. The first protrusion 222 may be positioned at a different location on the distal face 211 compared to the second protrusion 240. For example, first protrusion 222 may be positioned generally circumferentially/diametrically opposite one another on distal face 211.

Housing 201 may include a working channel opening 216, an electronics channel opening 218, and a fluid channel opening 220. Each of working channel opening 216, electronics channel opening 218, and fluid channel opening 220 may be formed on distal face 211. In some aspects, working channel opening 216 may be fluidly connected to a working channel of a shaft, such as shaft 104. Electronics channel opening 218 may be in communication with a shaft through which electronics (e.g., wiring or cabling) extend. The fluid channel opening 220 may be fluidly connected to one or more fluid channels of the shaft. Each of working channel opening 216, electronics channel opening 218, and fluid channel opening 220 may be distal openings of a respective channel that is defined through housing 201 and connected respective lumens, channels, or conduits (not shown) defined through the shaft. For example, an electronics channel 219 (FIG. 6A) may extend from a proximal end of housing 201 to distal face 211.

Each of openings 216, 218, 220 may be positioned in different locations on distal face 211. For example, working channel opening 216 may be positioned between the first protrusion 222 and the second protrusion 240. In some examples, the first protrusion 222 and the second protrusion 240 may extend distally from distal face 211 on opposite sides of working channel opening 216. Electronics channel opening 218 may be positioned laterally on a portion of distal face 211 (e.g., at a distal end of electronics channel 219) from which second protrusion 240 extends. As will be discussed in detail below, second protrusion 240 may include one or more side walls 242 that include a lateral wall 246 positioned between working channel opening 216 and electronics channel opening 218. Fluid channel opening 220 may be positioned above (e.g., nearer to top side 201a than) electronics channel opening 218. Fluid channel opening 220 may also be laterally aligned with second protrusion 240.

Although not shown in FIG. 2-7B, housing 201 may include an elevator (e.g., elevator 146) (FIG. 1). An elevator channel 212 of distal tip 200 sized and shaped to receive at least a portion of the elevator and may be defined between first protrusion 222 and second protrusion 240. As shown, elevator channel 212 may be positioned distally to and generally aligned with working channel opening 216. The elevator may be configured to receive and contact an accessory device (not shown) extending distally from working channel opening 216 into elevator channel 212.

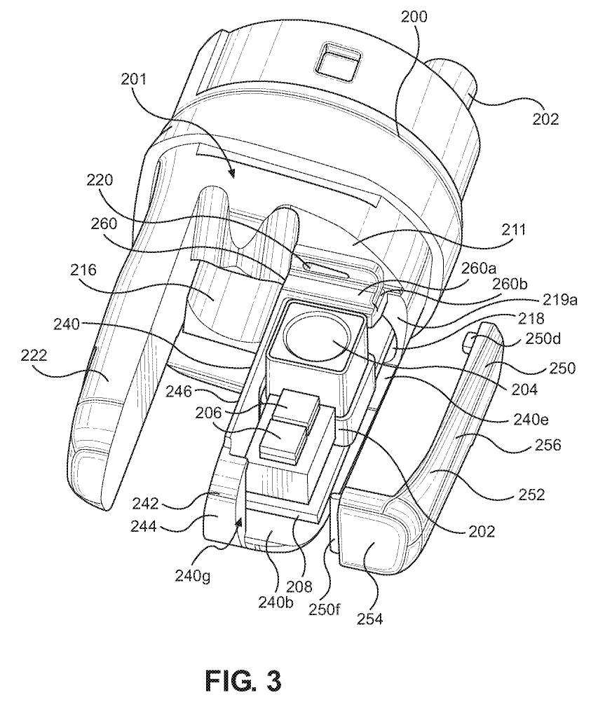

Housing 201 may include an electronics chamber 230. Electronics chamber 230 may be sized and shaped to receive an electronics assembly 202. Electronics chamber 230 may be positioned distally relative to electronics channel opening 218 and may be generally aligned with electronics channel opening 218 in some configurations. For example, electronics assembly 202 may be extended through electronics channel 219 of housing 201 and into shaft 104 via electronics channel opening 218. At least a portion of electronics assembly 202 may extend into electronics chamber 230. Second protrusion 240 may at least partially define the boundaries of electronics chamber 230. For example, one or more side walls 242 may include a distal wall 244 extending from a distal end of a lateral wall 246. Distal wall 244 may extend laterally outwardly, away from elevator channel 212. Distal wall 244 may have a thickness in the proximal/distal direction, such that a distal face of distal wall 244 is distal to a distal end of lateral wall 246. The combination of distal wall 244, lateral wall 246, and a base surface 240b or base wall (see, e.g., FIG. 3) may at least partially define the boundaries of electronics chamber 230. Electronics chamber 230 may form a partially enclosed space that is suitable for housing sensitive electronic components of electronics assembly 202.

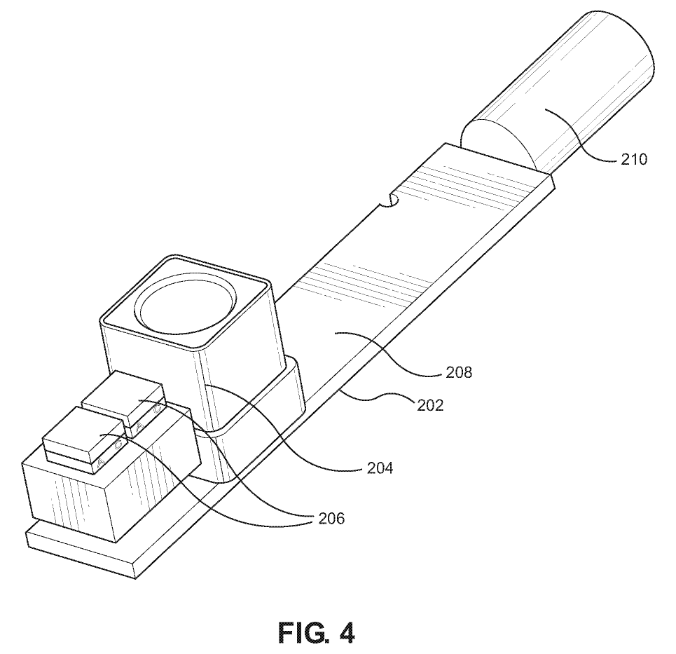

FIG. 4 depicts a perspective view of electronics assembly 202. Electronics assembly 202 may be configured to be compact and modular, allowing for easy installation within electronics chamber 230. Electronics assembly 202 may include one or more electrical components such as, but are not limited to: imagers (e.g., cameras), lighting elements (e.g. LEDs), power management integrated circuits, analog-to-digital converters, digital signal processors, temperature sensors, pressure sensors, accelerometers, gyroscopes, wireless communication modules (e.g., Bluetooth, Wi-Fi), voltage regulators, capacitors, resistors, transistors, and connectors or terminals. For example, electronics assembly 202 may include an imager 204 and a lighting element 206. When installed in electronics chamber 230, imager 204 and lighting element 206 may face generally radially outward (may be side-facing, as discussed above), which may be a top/upward direction, as described above.

A circuit board 208 of electronics assembly 202 may extend longitudinally, approximately parallel to a longitudinal axis of the shaft (e.g., shaft 104) or a longitudinal axis of distal tip 200. Electronics assembly 202 may include a conduit 210 for receiving other cables, wires, etc., or may be a tube or cable. Conduit 210 may be flexible or rigid. Conduit 210 may include appropriate electrical connections for each of the one or more components of electronics assembly 202. For example, conduit 210 may include one or more electrical conduits (e.g., wires or cables) for providing electrical power to lighting element 206 and one or more electrical conduits (e.g., wires or cables) for providing electrical power and electrical signals to imager 204 or receiving electrical signals from imager 204. The circuit board 208 may provide a mounting surface for electronic components and provide electrical connections between, for example, the imager 204, lighting element 206, and the conduit 210. Conduit 210 extends from an end (e.g., a proximal end) of the circuit board 208. Electronics assembly 202 may be positioned within electronics chamber 230 such that imager 204 and lighting element 206 face radially outward, perpendicularly, approximately perpendicularly, or otherwise transverse to a central longitudinal axis of distal tip 200 (e.g., at an angle of approximately 80 degrees to an angle of approximately 110 degrees), referred to herein as axis A (FIG. 6C). In other words, imager 204 and lighting element 206 may face upwardly, using the coordinate system described above.

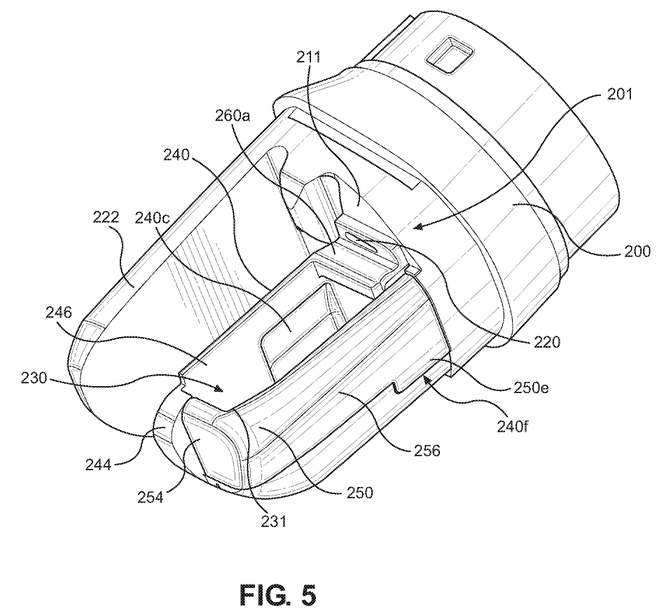

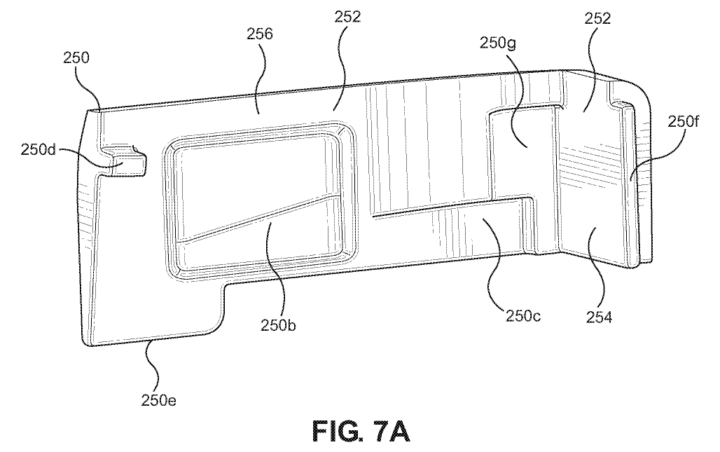

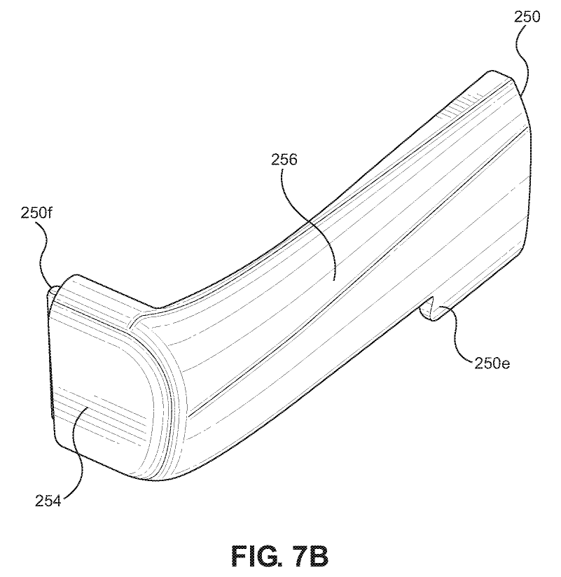

Distal tip 200 may include a dam 250 that, in conjunction with second protrusion 240 of housing 201, defines electronics chamber 230. Dam 250 may be removably or fixedly couplable to second protrusion 240. Accordingly, distal tip 200 may include an assembled state when dam 250 is coupled to housing 201 (e.g., second protrusion 240) and an unassembled state when dam 250 is not coupled to housing 201. In FIG. 2, dam 250 is connected to housing 201 to define electronics chamber 230, and electronics assembly 202 is in electronics chamber 230. In FIG. 3, dam 250 is shown as disconnected from housing 201, and electronics assembly 202 is positioned within housing 201. In FIG. 5, dam 250 is coupled to housing 201 including second protrusion 240 and electronics assembly 202 is not shown, in order to illustrate features of electronics chamber 230. FIGS. 6A-6C depict housing 201 but not dam 250, so as to show features of housing 201. FIGS. 7A and 7B show dam 250 in isolation to show details of dam 250.

Dam 250 may include one or more walls 252 that form part of the boundaries of electronics chamber 230. For example, dam 250 may include a dam lateral wall 256 (a side wall) and a dam distal wall 254 that, together with lateral wall 246, distal wall 244, and base surface 240b of second protrusion 240, and a portion of distal face 211, define electronics chamber 230. Dam 250 may be formed from a plastic, polymer, or similar material. Electronics chamber 230 may include an opening 231. Opening 231 may be positioned at a top end or portion (e.g., an end or portion nearest top side 201a of distal tip 200) of electronics chamber 230, which may be at a top side of second protrusion 240 or dam 250. Opening 231 may be in fluid communication with the electronics channel 219 and electronics channel opening 218.

When dam 250 is coupled to second protrusion 240, surfaces of dam 250 abutting or contacting surfaces of second protrusion 240 may form fluid-tight seals therebetween. Accordingly, electronics chamber 230 may be filled with an encapsulation material 232. Encapsulation material 232 may at least partially surround electronics assembly 202. The encapsulation material 232 may be introduced into electronics chamber 230 through a proximal end of electronics channel 219 (e.g., such that it flows distally through opening 218 into chamber 230). Encapsulation material 232 may alternatively or additionally be introduced into electronics chamber 230 via opening 231 or another suitable access point. A presence of encapsulation material 232 at opening 231 and the proximal end of electronics channel 219 may help to form fluid-tight seals at opening 231 and at the proximal end of electronics channel 219 to prevent fluid flow past encapsulation material 232. For example, when cured, encapsulation material 232 form a material surface at opening 231.

While introducing encapsulation material 232, encapsulation material 232 may flow around and envelop the components of electronics assembly 202 positioned within electronics chamber 230. Encapsulation material 232 may help to fix a position of electronics assembly 202 within electronics chamber 230. Further, encapsulation material 232 may be configured to fluid/water-proof and electrically insulate one or more components of electronics assembly 202. Encapsulation material 232 may be configured to prevent fluids from entering electronics channel opening 218 and electronics chamber 230. The encapsulation material 232 may include various substances such as epoxy resins, silicone compounds, or other polymeric materials suitable for electronic component encapsulation. In some cases, the encapsulation material 232 may be selected based on its thermal conductivity, electrical insulation properties, or resistance to moisture and environmental factors. In some aspects, encapsulation material 232 may be selected based on transparency. Encapsulation material 232 may be transparent or substantially transparent so that all or a substantial portion of light emitted by lighting element 206 or received by imager 204 is not obstructed or absorbed by encapsulation material 232 between material surface 234, and imager 204 or lighting element 206. In some aspects, encapsulation material 232 may cover an outer/top surface of imager 204 or lighting element 206. Explained differently, the outer/top surface of imager 204 or lighting element 206 may be positioned below material surface 234 of encapsulation material 232. Alternatively, encapsulation material 232 may only cover the outer/top surface of lighting element 206 and may not cover the outer/top surface of imager 204. In other words, the outer/top surface of imager 204 may be positioned above material surface 234 and the outer/top surface(s) of lighting element 206 may be positioned below material surface 234.

As shown at least in FIGS. 2-3, distal tip 200 may include a ramp 260. Ramp 260 may be positioned below fluid channel opening 220. Fluid (e.g., irrigation fluid) may flow distally out of opening 220 and flow distally across ramp 260. The flow of fluid across ramp 260 may help to clean or remove debris from imager 204. For example, fluid may flow across an angled surface 260a (see, e.g., FIG. 3) of ramp 260. Angled surface 260a may extend distally from distal face 211. Ramp 260, including angled surface 260a, may be integrally formed with second protrusion 240 or distal face 211.

Angled surface 260a may slope upward from a proximal end to a distal end (FIG. 6B) of angled surface 260a. In some aspects, angled surface 260a may slope downward from the proximal end to the distal end. Angled surface 260a may be configured such that the distal end of angled surface 260a is aligned or generally aligned with a top surface of imager 204 or material surface 234 (e.g., a top surface) of encapsulation material 232. Explained differently, a length (e.g., parallel to axis A) of angled surface 260a along the longitudinal axis of distal tip 200 may be sized to extend from opening 220 to a proximal end of the outer/top surface or lens of imager 204 or a proximal end of material surface 234 of encapsulation material 232 such that fluid may flow from opening 220, across angled surface 260a, and across the outer/top surface of imager 204 or encapsulation material 232. In some aspects, a width of angled surface 260a may be wider (e.g., relative to an axis perpendicular to axis A) than fluid channel opening 220 to accommodate fluid flow. As shown in FIG. 3, the angle of inclination may vary depending on the desired fluid flow characteristics. For example, the angle may range from about 5 degrees to about 60 degrees. In some aspects, angled surface 260a may have a curved or contoured profile rather than a strictly linear slope. In such aspects, the angled surface 260a may include a concave curve. This curvature may help direct fluid flow in a specific pattern or trajectory. Angled surface 260a may be smooth to promote laminar fluid flow, or it may include texture or structures to create turbulence or specific flow patterns.

Referring to FIGS. 2 and 6A-6C, ramp 260 may include one or more structures to help to facilitate coupling of dam 250 to second protrusion 240. For example, ramp 260 may include a ramp recess 260b positioned at a laterally outward end of ramp 260. Ramp recess 260b may be configured (e.g., sized, shaped or otherwise capable of) to receive a corresponding portion of dam 250 to couple dam 250 to second protrusion 240.

Features of second protrusion 240 and dam 250 will now be discussed in further detail. FIGS. 6A-6C depict various views of second protrusion 240, FIGS. 7A-7B depict various views of dam 250, and FIG. 5 depicts dam 250 coupled to second protrusion 240. Referring now to FIGS. 6A-6C, as discussed above, second protrusion 240 may extend distally from distal face 211 and may include one or more side walls 242 and base surface 240b. One or more side walls 242 may extend upward (e.g., toward top side 201a of distal tip 200) from base surface 240b. One or more side walls 242 includes lateral wall 246 and distal wall 244. Lateral wall 246 may extend longitudinally relative to the longitudinal axis of distal tip 200 from distal face 211 to a distal end of lateral wall 246. In other words, lateral wall 246 may extend distally from distal face 211 and may extend generally parallel to the longitudinal axis of distal tip 200 (axis A) to the distal end of lateral wall 246. Distal wall 244 may extend laterally outward from the distal end of lateral wall 246 relative to axis A. Explained differently, distal wall 244 may extend generally perpendicular relative to a longitudinal axis (axis A) of distal tip 200 toward an exterior surface of housing 201. Distal wall 244 may extend along a portion of a distal edge of base surface 240b and may terminate prior to a midpoint of the distal edge of base surface 240b. A distance between a distal end of distal wall 244 and distal face 211 may be approximately a same distance as between a distal end of first protrusion 222 and distal face 211.

Interior surfaces of distal wall 244 and lateral wall 246 (surfaces facing electronics chamber 230) may be generally planar but may include one or more cavities or depressions configured to receive encapsulation material 232 or portions of electronics assembly 202. It should be understood that references to interior surfaces of second protrusion 240 and dam 250 refer to surfaces forming boundaries of electronics chamber 230. The one or more cavities or depressions of distal wall 244 and lateral wall 246 may help to prevent translation, rotation, and vibration of electronics assembly 202 or encapsulation material 232 within electronics chamber 230. For example, lateral wall 246 may include a proximal cavity 240c (FIGS. 5-6C) and a distal cavity 240d (FIGS. 6A-6C).

Each of the proximal cavity 240c and distal cavity 240d may be configured to provide clearance for a portion(s) of electronics assembly 202 or encapsulation material 232. Clearance for encapsulation material 232 may permit encapsulation material 232 to flow around the respective portion(s) of electronics assembly 202 to secure electronics assembly 202 (and portions thereof) within chamber 230. For example, proximal cavity 240c may be configured to provide clearance for imager 204 or encapsulation material 232 on, contacting, or adjacent to electronics assembly 202 (e.g., imager 204 or the lens of imager 204). Proximal cavity 240c may be at least partially aligned with imager 204. Distal cavity 240d may be configured to provide clearance for lightning element(s) 206 or circuit board 208, or encapsulation material 232 on, contacting, or adjacent to electronics assembly 202 (e.g., lighting element(s) 206 or a distal end of circuit board 208). Distal cavity 240d may be at least partially aligned with lighting element(s) 206. Distal cavity 240d may be positioned distally to proximal cavity 240c.

Proximal cavity 240c may extend from base surface 240b toward the top end of lateral wall 246. Proximal cavity 240c may include a generally rectangular shape. A top portion of proximal cavity 240c may taper toward the top end of lateral wall 246. Explained different, the depth of the top portion of proximal cavity 240c into lateral wall 246 may decrease from a bottom end of the top portion to a top end of the top portion of proximal cavity 240c.

Distal cavity 240d may extend from base surface 240b and extend from distal wall 244. Distal cavity 240d may extend from base surface 240b toward the top end of lateral wall 246. Compared to distal cavity 240d, the top end of proximal cavity 240c may be nearer to the top end of lateral wall 246 than a top end of distal cavity 240d is. Distal cavity 240d may also extend from distal wall 244 toward the proximal end of housing 201. Similar to proximal cavity 240c, distal cavity 240d may include a generally rectangular shape. At least a portion of a perimeter of proximal cavity 240c or distal cavity 240d may be curved.

Base surface 240b may form a bottom portion of second protrusion 240. In other words, base surface 240b may be positioned at bottom portions or ends of distal wall 244 and lateral wall 246. Base surface 240b may extend longitudinally, approximately parallel to the longitudinal axis (axis A) of distal tip 200. A top side of base surface 240b may be generally planar and configured to support at least a portion of electronics assembly 202. Base surface 240b may include a recess 240e positioned at a proximal end of second protrusion 240. Recess 240e may help to provide a mounting location for electronics assembly 202 or serve as a clearance space.

Recess 240e may extend radially inward (e.g., generally perpendicular to axis A) from a laterally outward edge of base surface 240b toward a laterally inward edge of base surface 240b (toward lateral wall 246). In some aspects, recess 240e may have a depth that varies along its length. For example, the depth of recess 240e may decrease from a proximal end of recess 240e to a distal end of recess 240e. The varying depth may provide additional clearance for components of electronics assembly 202. In some configurations, recess 240e may include one or more surface features, such as grooves, ridges, curves, or textures, that may enhance adhesion of encapsulation material 232 or provide additional clearance. For example, at least a portion of the perimeter of recess 240e may be curved.

As shown particularly in FIGS. 6A-6C, second protrusion 240, including distal wall 244, lateral wall 246, and base surface 240b, may include one or more structures or features to help to facilitate coupling of dam 250 to second protrusion 240. For example, distal wall 244 may include a wall recess 240g configured to receive a portion of dam 250 to couple dam 250 to second protrusion 240. Wall recess 240g may extend laterally inward relative to axis A from a laterally outward end of distal wall 244. Wall recess 240g may be positioned at a proximal end of a laterally outward surface of distal wall 244.

Base surface 240b may include a proximal locking channel 240f configured to receive or be coupled to at least a portion of dam 250. Proximal locking channel 240f may be in communication with recess 240e and may be an open laterally outward side of recess 240e. Relative to axis A (e.g., the central longitudinal axis of distal tip 200) proximal locking channel 240f may be a radially outwardmost portion of recess 240e (e.g., at a radially outward edge of base surface 240b). A distal end of proximal locking channel 240f may have a curved shape. The curved shape of proximal locking channel 240f may help to secure dam 250 in place when coupled to second protrusion 240 because dam 250 may have a surface with a complementary shape, as described below. For example, proximal locking channel 240f may help to limit or prevent proximal and distal movement of dam 250 relative to second protrusion 240. In some aspects, at least a portion of a proximal edge of recess 240e may be aligned with a portion of a perimeter of electronics channel opening 218. This alignment may help to guide the insertion of conduit 210 into electronics channel 219. In some aspects, the proximal edge of recess 240e may include a contoured or shaped section that transitions to electronics channel opening 218, allowing for a smooth transition between electronics channel opening 218 and the electronics chamber 230. In some examples, this alignment of recess 240e and may create a continuous surface from the electronics channel opening 218 to the electronics chamber 230, which may facilitate the flow of encapsulation material 232 during the filling process.

Now referring to FIGS. 7A and 7B, as discussed above, dam 250 may include one or more walls 252 which may include dam lateral wall 256 and dam distal wall 254. Dam distal wall 254 may be positioned at a distal end of dam lateral wall 256 and extend radially inward (e.g., perpendicular or generally perpendicular) relative to axis A from the distal end of dam lateral wall 256. Accordingly, dam 250 may include or have a substantially “L”-shape. The substantially “L”-shape of dam 250 may correspond to a substantially “L” shape of a portion of the perimeter of second protrusion 240 not occupied by lateral wall 246 and distal wall 244. Dam 250 may be coupled (with or without use of an adhesive) to second protrusion 240 so that dam lateral wall 256 is parallel or generally parallel to axis A and dam distal wall 254 is perpendicular or generally perpendicular to axis A. Explained differently, dam 250 may be coupled to second protrusion 240 such that dam lateral wall 256 extends along the laterally outward (e.g., laterally outwardmost) edge of base surface 240b and dam distal wall 254 extends along a distalmost edge of base surface 240b.

FIG. 7A generally shows interior surfaces of dam 250 (e.g., the portion of dam 250 defining boundaries of electronics chamber 230) and FIG. 7B generally shows exterior surfaces of dam 250. Interior surfaces of dam distal wall 254 and dam lateral wall 256 may be generally planar but may include one or more features to help to facilitate coupling of dam 250 to second protrusion 240. For example, the one or more features of dam 250 may include one or more cavities or depressions configured to receive encapsulation material 232 or portions of electronics assembly 202 and one or more protrusions configured to be received within corresponding recesses of distal tip 200 (e.g., recesses of second protrusion 240 and ramp 260). Similar to second protrusion 240, the one or more cavities or depressions of dam distal wall 254 and dam lateral wall 256 may help to prevent translation, rotation, and vibration of electronics assembly 202 or encapsulation material 232 within electronics chamber 230.

The one or more features of dam 250, including dam distal wall 254 and dam lateral wall 256, for receiving encapsulation material 232 or providing clearance a portion of electronics assembly 202. For example, the one or more features of dam 250 may include a proximal cavity 250b and a distal cavity or channel 250c. Proximal cavity 250b may be sized and shaped to receive encapsulation material 232 contacting imager 204 or provide clearance for electronics module 202/imager 204. In other words, proximal cavity 250b may be at least partially aligned with imager 204 and may allow for encapsulation material 232 to flow around imager 204 and secure imager 204 within chamber 230. Proximal cavity 250b may have a substantially rectangular shape. A perimeter of proximal cavity 250b may be curved. Both a top end and a bottom end of proximal cavity 250b may be positioned between a top end of dam lateral wall 256 and a bottom end of dam lateral wall 256. Distal channel 250c may provide clearance for electronics assembly 202 within electronics chamber 230. Distal channel 250c may include a top wall and a distal wall. A depth of the top wall of distal channel 250c, in a direction perpendicular to axis A, may increase in the proximal-to-distal direction. Explained differently, an interior, planar surface of distal channel 250c may be angled away from axis A in the proximal-to-distal direction.

Still referring to FIG. 7A, dam 250 may include a recess 250g configured to receive or provide clearance at least a portion of electronics module 202. In particular, recess 250g may be configured to provide clearance for one or more lighting elements 206. One or more lighting elements 206 may include any size, shape, or geometry. Accordingly, recess 250g may be sized and shaped to receive one or more lightning elements 206 of varying sizes, shapes, and geometries. Recess 250g may include a planar surface. Recess 250g may share an edge with the interior surface of dam lateral wall 256. For example, recess 250g may share a radially outward edge of the interior surface of dam lateral wall 256. Further, recess 250g may share a distal edge or surface with distal channel 250c. Recess 250g may extend from a bottom edge of dam 250. A distal portion of distal channel 250c may extend longitudinally, approximately parallel to the longitudinal axis (axis A) of distal tip 200, through a portion of recess 250g. When viewed from the viewing angle of FIG. 7A, recess 250g may have an approximately “P”-shape without the counter (e.g., the area of a letter that is entirely or partially enclosed by a letter form or symbol).

Further, the one or more features of dam 250, including dam distal wall 254 and dam lateral wall 256, may include one or more structures or features to help to facilitate coupling of dam 250 to second protrusion 240. For example, the one or more features of dam 250 may include a first proximal protrusion 250d. Proximal protrusion 250d may extend radially inward or perpendicular relative to axis A from the interior surface of dam lateral wall 256. Proximal protrusion 250d may be disposed near or proximate a top edge of dam 250. Proximal protrusion 250d may be at (e.g., contacting) a proximalmost edge of dam lateral wall 256. When dam 250 is coupled to second protrusion 240, proximal protrusion 250d may be positioned within ramp recess 260b.

The one or more features of dam 250 may include a second protrusion 250e. Proximal protrusion 250e may extend distally from the proximal end of dam lateral wall 256 and may extend downward from the bottom end or edge of dam lateral wall 256. Second protrusion 250e may be sized and shaped to engage with or be received within proximal locking channel 240f of second protrusion 240. Accordingly, when dam 250 is coupled to second protrusion 240, second locking protrusion 250e may be received within or engaged with proximal locking channel 240f or recess 240e.

The one or more features of dam 250 may further include a distal protrusion 250f. Distal protrusion 250f may be positioned at a laterally inward end of dam distal wall 254, in a direction facing toward and approximately perpendicularly to axis A. Distal protrusion 250f may be positioned at a proximal end of a laterally inward surface of dam distal wall 254. Distal protrusion 250f may extend laterally inward or perpendicular to axis A. Distal protrusion 250f may be sized and shaped to be received within wall recess 240g. Thus, when dam 250 is coupled to second protrusion 240, distal protrusion 250f may be received within wall recess 240g.

The overall shape and configuration of dam 250 may be designed to complement the geometry of second protrusion 240 and other components of distal tip 200. For example, when in the assembled state, the interior surface of dam lateral wall 256 of dam 250 may contact a curved surface 219a (FIG. 3) of electronics channel 219 extending distally beyond distal face 211. In some aspects, the interior surface of dam lateral wall 256 may include a curve complementary or corresponding to curved surface 219a of electronics channel 219. When assembled, dam 250 and second protrusion 240 may form a cohesive structure that effectively encloses and protects electronics assembly 202 while allowing for the introduction and curing of encapsulation material 232.

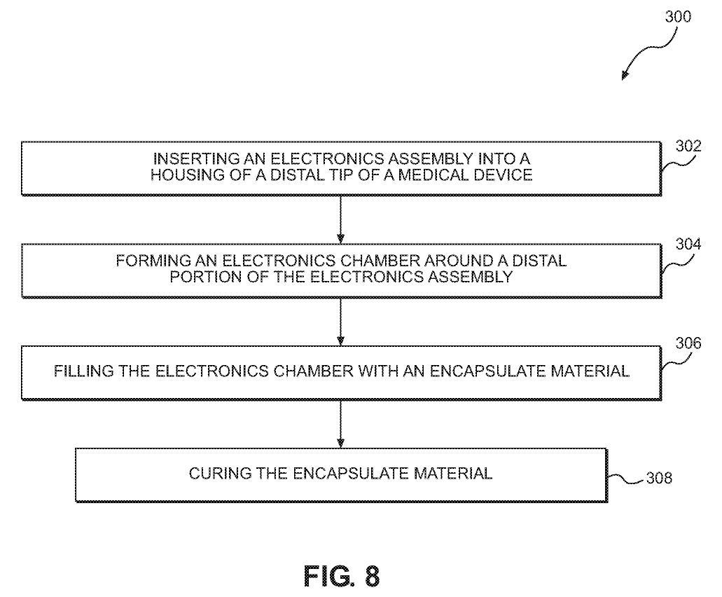

FIG. 8 depicts a flow chart of a method of manufacturing an exemplary distal tip (e.g., distal tip 130 or distal tip 200) of an exemplary medical device, a method 300. Method 300 may include a step 302 of inserting an electronics assembly (e.g., electronics assembly 202) into a housing (e.g., housing 201) of a distal tip (e.g., distal tip 200) of a medical device (e.g., medical device 100). For example, while dam 250 is removed or not coupled to second protrusion 240 (e.g., distal tip 200 is in the unassembled state), a portion of electronics assembly 202 may be inserted or extended through electronics channel opening 218 and electronics channel 219. Explained differently, conduit 210 or a portion of circuit board 208 may be inserted or extended proximally through electronics channel opening 218 and electronics channel 219. A cross-section or dimension of electronics channel opening 218 may be smaller than a cross-section or dimension of imager 204 such that imager 204 may not be inserted or extended through opening 218. Further during or after insertion, while dam 250 is removed or not coupled to second protrusion 240, electronics assembly 202 may be positioned. Moreover, the absence of dam 250 while positioning or inserting electronics assembly 202, may help to ease positioning or insertion by not obstructing electronics channel opening 218 or otherwise blocking access to electronics chamber 230. For example, one or more dimensions of electronics assembly 202 may be larger than one or more dimensions of opening 231 when dam 250 is assembled with housing 201, or dimensions of electronics assembly 202 may otherwise prevent electronics assembly 202 from being inserted with dam 250 in place. After insertion, electronics assembly 202 may be positioned such that each of imager 204 and lighting element 206 is distal to distal face 211. Imager 204 may be positioned proximal to lighting element 206 along a longitudinal axis of electronics assembly 202.

Method 300 may further include a step 304 of forming an electronics chamber (e.g., electronics chamber 230) around a distal portion of the electronics assembly (e.g., electronics assembly 202). The distal portion of the electronics assembly may include imager 204 and lighting element 206 or may include a portion of electronics assembly 202 distal to distal face 211. Step 304 may include coupling one or more coupling portions of dam 250 to one or more corresponding coupling portions of second protrusion 240 to form electronics chamber 230. When electronics chamber 230 is formed (e.g., dam 250 is coupled to housing 201) distal tip 200 may be in the assembled state. Coupling and coupling portions of second protrusion 240 and electronics chamber 230 are discussed in further detail above.

Method 300 may include a step 306 of filling the electronics chamber (e.g., electronics chamber 230) with an encapsulation material (e.g., encapsulation material 232). Encapsulation material 232 may be introduced into electronics chamber 230 via the proximal end of electronics channel 219 or introduced into electronics chamber 230 via opening 231. While filling electronics chamber 230, encapsulation material 232 may adopt the shape of electronics chamber 230 including cavities, channels, recesses, holes, openings, surfaces, and so on, of interior surfaces of second protrusion 240, dam 250, and distal face 211, and may encapsulate, encase, envelop, or otherwise wholly surround a portion of electronics assembly 202 positioned within electronics chamber 230.

Encapsulation material 232 may form a material surface 234 at opening 231. In some configurations, the material surface 234 may be shaped or contoured to align with the geometry of opening 231. For example, the material surface 234 may be formed with a slight convex or concave profile to enhance its sealing capabilities or to direct light from lighting element 206 in a desired pattern. As shown in FIG. 2, a top end or portion of imager 204 may be positioned above material surface 234. Positioning the top end or portion of imager 204 may help to prevent the field of view of imager 204 from being obscured by encapsulation material 232 and may allow a lens of imager 204 to be cleaned or washed via fluid channel opening 220. As discussed above, another material surface of encapsulation material 232 may be formed at the proximal end of electronics channel 219. The material surface at the proximal end of electronics channel 219 and material surface 234 may help to prevent fluid flow (e.g., bodily fluids or irrigation fluid) through electronics channel 219 (e.g., via the proximal end of electronics channel 219) and opening 231 of electronics chamber 230. In other words, the material surfaces may fluidly isolate a space between material surface 234 and the material surface at the proximal end of electronic channel 219.

Still referring to FIG. 8, method 300 may include a step 308 of curing the encapsulation material (e.g., encapsulation material 232). In some aspects, curing may involve exposing encapsulation material 232 to heat, ultraviolet light, or allowing it to cure at room temperature for a predetermined time period. The curing process may help solidify the encapsulation material 232, including material surface 234 and the material surface at the proximal end of electronics channel 219, potentially improving its protective properties. In some cases, the curing step may be performed in stages, with an initial partial cure followed by a final cure. The curing parameters, such as temperature, duration, and curing method, may be selected based on the specific properties of the encapsulation material 232 used and the desired characteristics of the final assembly.

While principles of this disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

What is claimed is:

1. A distal tip of a medical device comprising:
a housing defining a distal opening of a working channel;
a dam having a lateral wall and a distal wall, wherein, in an unassembled state of the distal tip, the dam is separate from the housing, wherein, in an assembled state of the distal tip, the dam and the housing define a chamber;
an electronics assembly disposed within the chamber in the assembled state of the distal tip, wherein the electronics assembly includes an imager and a lighting element; and
an encapsulation material within the chamber and at least partially surrounding the electronics assembly.

2. The distal tip of claim 1, wherein the dam includes a protrusion that is received by a recess of the housing in the assembled state.

3. The distal tip of claim 1, wherein, in the assembled state, a proximal portion of the electronics assembly extends into a channel of the housing, and wherein the channel has a smaller cross-section than a cross-section of the imager.

4. The distal tip of claim 1, wherein the dam has a substantially “L”-shape.

5. The distal tip of claim 1, wherein the chamber has a top opening, and wherein the imager and the lighting element face outward, through the top opening.

6. The distal tip of claim 1, wherein the housing includes a ramp positioned proximal of the chamber.

7. The distal tip of claim 6, wherein the housing defines a fluid channel opening positioned above the chamber, and wherein the ramp is configured to guide a flow of fluid from the fluid channel opening across a lens of the imager or across the encapsulation material.

8. The distal tip of claim 7, wherein the ramp includes an angled surface that is angled upward in a distal direction.

9. The distal tip of claim 1, wherein the housing includes a protrusion, the protrusion at least partially defining the chamber.

10. The distal tip of claim 9, wherein the protrusion includes a base and one or more side walls.

11. The distal tip of claim 10, wherein a side wall of the one or more side walls includes a first recess at least partially aligned with the imager.

12. The distal tip of claim 11, wherein a side wall of the one or more side walls includes a second recess at least partially aligned with the lighting element.

13. The distal tip of claim 10, wherein a proximal end of the base includes a recess.

14. The distal tip of claim 9, wherein the protrusion is a first protrusion, wherein the housing includes a second protrusion, and wherein the first protrusion and the second protrusion define a channel therebetween for an elevator.

15. The distal tip of claim 1, wherein the dam includes a recess at least partially aligned with the imager.

16. A distal tip for a medical device comprising:
a housing including a distal face;
a first protrusion and a second protrusion extending distally from the distal face;
a dam coupled to the second protrusion, the dam and the second protrusion together defining boundaries of an electronics chamber; and
an electronics assembly positioned within the electronics chamber.

17. The distal tip of claim 16, wherein:
the second protrusion includes a base surface and one or more side walls extending from the base surface;
the dam includes one or more walls; and
at least one of the one or more walls of the dam includes a first feature configured to engage with a second feature of the one or more side walls of the second protrusion to form the boundaries of the electronics chamber.

18. A method for manufacturing a distal tip of a medical device comprising:
inserting an electronics assembly into a housing of the distal tip of the medical device;
forming an electronics chamber around a distal portion of the electronics assembly by coupling a dam to the housing;
filling the electronics chamber with an encapsulation material; and
curing the encapsulation material.

19. The method of claim 18, wherein forming the electronics chamber comprises coupling the dam to a protrusion of the distal tip.

20. The method of claim 18, wherein the encapsulation material forms a fluid-tight seal between at an opening of the electronics chamber and an opening of an electronics channel of the housing.
